(19) United States
(12) Patent Application Publication
(10) Pub. No.: US 2005/0188479 A1
(43) Pub. Date: Sep. 1, 2005

(54) Compositions for dyeing keratin fibers comprising an alcohol oxidase and a polyurethane associative polymer and processes using the composition

(76) Inventor: Gregory Plos, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/044,275

(22) Filed: Jan. 28, 2005

Related U.S. Application Data
(60) Provisional application No. 60/545,482, filed on Feb. 19, 2004.

(30) Foreign Application Priority Data
Jan. 28, 2004 (FR) 04 00779

(51) Int. Cl. A61K 7/13

(52) U.S. Cl. 8/405

(57) ABSTRACT

The disclosure provides compositions for dyeing keratin fibers, such as human keratin fibers including the hair, comprising, in a medium that is suitable for dyeing, at least one oxidation dye precursor, at least one alcohol oxidase enzyme, at least one enzyme substrate, and at least one polyurethane associative polymer. The disclosure also provides processes for dyeing keratin fibers, which comprises applying the compositions, and also to dyeing “kits.”

This application claims the benefit under 35 U.S.C. § 119 of U.S. Provisional Application No. 60/545,482, filed Feb. 19, 2004, and French Application FR 04/00779, filed Jan. 28, 2004, both of which are hereby incorporated by reference.

The present disclosure relates to compositions for dyeing keratin fibers, including human keratin fibers such as the hair, comprising, in a medium suitable for dyeing, at least one oxidation dye precursor, at least one alcohol oxidase enzyme, at least one enzyme substrate, and at least one polyurethane associative polymer.

It is a known practice to dye keratin fibers, and in particular human hair, with dye compositions comprising oxidation dye precursors, such as ortho- and para-phenylenediamines, ortho- and para-aminophenols, and heterocyclic compounds, which are all generally referred to as oxidation bases. These oxidation bases are colorless or weakly colored compounds which, when combined with oxidizing products, give rise to colored compounds by a process of oxidative condensation.

It is also known that the shades obtained with these oxidation bases may be varied by combining them with couplers or coloration modifiers such as aromatic meta-diamines, meta-aminophenols, meta-diphenols, and certain heterocyclic compounds such as indole compounds.

The variety of molecules used as oxidation bases and couplers makes it possible to obtain a wide range of colors.

The permanent coloration obtained using these oxidation dyes should also satisfy certain requirements: it should, for example, have no toxicological drawbacks, allow shades of the desired intensity to be obtained, and/or have good resistance to external agents (e.g., light, bad weather, washing, permanent waving, perspiration, and rubbing).

Oxidation dyes ideally also allow white hair to be covered and they further should be as unselective as possible, i.e., allow the smallest possible difference in coloration to be produced over the entire length of the same keratin fiber, which is typically differently sensitized (e.g., damaged) between its end and its root.

The dyeing is typically performed in strongly alkaline medium, in the presence of hydrogen peroxide. However, the use of alkaline media in the presence of hydrogen peroxide has the drawbacks of causing considerable degradation of the fibers and bleaching of the keratin fibers, neither of which is particularly desirable.

Oxidation dyeing of keratin fibers may also be performed using oxidizing systems other than hydrogen peroxide, such as enzymatic systems. For example, French Patent Application 2,769,219 describes the use of a uricase enzyme and of its uric acid substrate in oxidation dyeing of keratin fibers. European Patent Application 310,675 A describes the use of benzenic oxidation dye precursors in combination with enzymes such as pyranose oxidase and glucose oxidase. In addition, French Patent Application 2,833,492 describes the use of an alcohol oxidase enzyme as the sole enzyme in an oxidation dye composition for dyeing keratin fibers.

The present disclosure provides novel thickened and stable compositions for dyeing keratin fibers by oxidation dyeing, using an oxidizing system other than hydrogen peroxide.

More specifically, the present inventor has discovered, advantageously and unexpectedly, that such compositions may be obtained by using at least one oxidation dye precursor, at least one alcohol oxidase enzyme, at least one enzyme substrate, and at least one polyurethane associative polymer in a composition for dyeing keratin fibers, including human keratin fibers such as the hair.

The compositions make it possible to obtain homogeneous formulations which, once applied, respect the nature of the keratin fibers and may not have the solubilization and crystallization problems often encountered with a uric acid/uricase system. Further, the stability of the compositions may be improved, including the stability of the alcohol oxidase enzyme.

The compositions disclosed herein may give dyeing results with strong, unselective and fast colors, and they may be capable of generating varied shades of intense and uniform color, without any significant degradation of the hair. In addition, the use of such compositions may also improve the hold of permanent-waved hair and reduce the porosity of the hair.

Further, the present inventor has also discovered that uptake of the dye onto the fibers advantageously is not stopped when the presently disclosed compositions are used. In contrast, the uptake of the dye generally does stop when dye compositions containing conventional thickeners, surfactants and solvents are used. When the uptake of the dye onto the fibers is stopped, a larger amount of dye must be used to produce a strong shade, and a larger amount of solvent and/or of surfactant is then required to dissolve this dye.

Other characteristics, aspects, subjects and advantages of the present invention will emerge even more clearly on reading the description and the examples that follow.

As used herein, the term “thickened composition” means a composition that can be applied to the area to be dyed without running outside of the area.

The viscosity of the compositions used may be greater than 200 cp, for example, greater than 500 cp, measured at 25° C. using a Rheomat RM 180 rheometer at a shear rate of 200 s⁻¹.

The associative polymers are chosen from water-soluble polymers, which in aqueous medium, are capable of reversibly combining together or with other molecules to lead to increased thickening of the medium.

The polyurethane associative polymers that may be used in the compositions comprise at least one terminal or pendent fatty chain having at least ten carbon atoms. These polymers are capable of interacting with themselves or with compounds of the medium in which they are present, such as surfactants, to lead to thickening of the medium. The polyurethane associative polymers may be cationic, anionic, or nonionic.

Cationic Associative Polyurethanes

Cationic polyurethane associative polymers that may be used include compounds in the family described in French Patent Application 0 009 609; this family of polymers may be represented by the general formula (I) below:
R—X—(P)ₙ—[L—(Y)ₘ]ᵣ—L′—(P′)ₚ—X′—R′ (I)
wherein:

R and R′, which may be identical or different, are each chosen from a hydrophobic group and a hydrogen atom;

X and X′, which may be identical or different, are each chosen from a group comprising an amine function optionally bearing a hydrophobic group, or alternatively a group L″;

L, L′ and L″, which may be identical or different, are each chosen from a group derived from a diisocyanate;

P and P′, which may be identical or different, are each chosen from a group comprising an amine function optionally bearing a hydrophobic group;

Y is a hydrophilic group;

r is an integer ranging from 1 to 100, for example, from 1 to 50 or from 1 to 25;

n, m and p each range, independently of each other, from 0 to 1000; and

wherein the polymer comprises at least one quaternary (e.g., protonated) or quaternized amine function and at least one hydrophobic group.

In some embodiments of these polyurethanes, the R and R′ groups at the chain ends are the only hydrophobic groups.

An example of a family of cationic associative polyurethanes that may be used in the presently disclosed compositions corresponds to formula (I) described above wherein R and R′ each independently represent a hydrophobic group, X and X′ each are L″, n and p are each integers ranging from 1 to 1000, and L, L′, L″, P, P′, Y and m are as described above.

Another family of cationic associative polyurethanes that may be used in the presently disclosed compositions is the family corresponding to formula (I) above wherein R and R′ each independently are chosen from a hydrophobic group, X and X′ each represent a group L″, n and p are each 0, and L, L′, L″, Y and m are as described above.

The fact that n and p are 0 means that these polymers do not comprise units derived from a monomer having an amine function, incorporated into the polymer during the polycondensation reaction. The protonated amine functions of these polyurethanes result from the hydrolysis of excess isocyanate functions, at the chain ends, followed by alkylation of the primary amine functions formed with alkylating agents having a hydrophobic group, i.e., compounds of the type RQ or R′Q, in which R and R′ are as defined above and Q denotes a leaving group such as, but not limited to, a halide or a sulfate.

Another family of cationic associative polyurethanes that may be used herein is the family corresponding to formula (I) above wherein R and R′ each independently are chosen from a hydrophobic group, X and X′ each independently are chosen from a group comprising a quaternary amine, n and p are 0, and L, L′, Y and m are as described above.

The number-average molecular mass of the cationic associative polyurethanes may range from 400 to 500,000, for example, from 1000 to 400,000 and from 1000 to 300,000.

As used herein, the term “hydrophobic group” means a radical or polymer comprising a saturated or unsaturated, linear or branched hydrocarbon-based chain, which may comprise one or more heteroatoms such as P, O, N, and S, or a radical comprising a perfluoro or silicone chain. When the hydrophobic group denotes a hydrocarbon-based radical, it comprises at least 10 carbon atoms, for example, from 10 to 30 carbon atoms, such as from 12 to 30 or even from 18 to 30 carbon atoms. In some embodiments, the hydrocarbon-based group may be derived from a monofunctional compound.

In some embodiments, the hydrophobic group may be derived from a fatty alcohol such as stearyl alcohol, dodecyl alcohol or decyl alcohol. In other embodiments, the hydrophobic group may be derived from a hydrocarbon-based polymer such as polybutadiene.

When X and/or X′ denote a group comprising a tertiary or quaternary amine, X is chosen from: 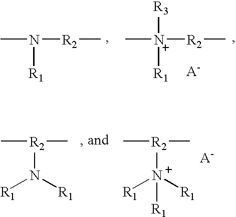
and X′ is chosen from: 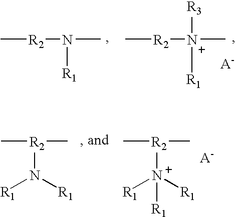
wherein:

R₂ is chosen from a linear or branched alkylene radical having from 1 to 20 carbon atoms, optionally comprising a saturated or unsaturated ring, and from an arylene radical, at least one of the carbon atoms optionally being replaced with a heteroatom chosen from N, S, O, and P;

R₁ and R₃, which may be identical or different, are each chosen from linear or branched C₁-C₃₀ alkyl radicals, linear or branched C₁-C₃₀ alkenyl radicals and aryl radicals, optionally comprising at least one (e.g., 1, 2 or 3) heteroatom chosen from N, S, O, and P; and

A⁻ is a physiologically acceptable counterion.

In some embodiments, the groups L, L′ and L″ represent a group of the formula: 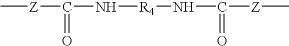
wherein:

Z is chosen from —O—, —S— or —NH—; and

R₄ is chosen from a linear or branched alkylene radical having from 1 to 20 carbon atoms, optionally comprising a saturated or unsaturated ring, and from an arylene radical, optionally comprising at least one (e.g., 1, 2 or 3) heteroatom such as N, S, O and P.

In some embodiments, the groups P and P′ comprise an amine function and are chosen from at least one of the formulae: 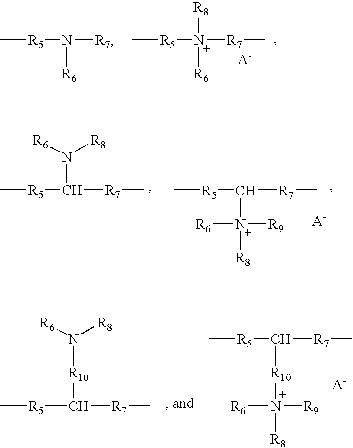
wherein:

R₅ and R₇, which may be identical or different, are each independently chosen from a linear or branched alkylene radical having from 1 to 20 carbon atoms, optionally comprising a saturated or unsaturated ring, and an arylene radical, at least one of the carbon atoms optionally being replaced with a heteroatom (e.g. 1, 2 or 3) chosen from N, S, O, and P;

R₆, R₈ and R₉, which may be identical or different, are each chosen from linear or branched C₁-C₃₀ alkyl radicals, linear or branched C₁-C₃₀ alkenyl radicals, and aryl radicals, optionally comprising at least one (e.g., 1, 2 or 3) heteroatom chosen from N, S, O, and P;

R₁₀ is chosen from a linear or branched, optionally unsaturated C₁-C₃₀ (e.g., C₁-C₂₀ or C₁-C₁₀) alkylene group optionally comprising at least one heteroatom (e.g., 1, 2 or 3) chosen from N, O, S and P; and

A⁻ is a physiologically acceptable counterion.

The term “hydrophilic group,” as used in the definition of Y, means a polymeric or non-polymeric water-soluble group.

In some embodiments, when the hydrophilic group is not polymeric, it may be a group derived from ethylene glycol, diethylene glycol, and propylene glycol.

In other embodiments, when the hydrophilic group is a hydrophilic polymer, it may be derived from, for example, polyethers, sulfonated polyesters, sulfonated polyamides, or a mixture of these polymers, for example, a polyether such as poly(ethylene oxide) or poly(propylene oxide).

The cationic associative polyurethanes of formula (I) that may be used may be formed from diisocyanates and from various compounds with functions comprising a labile hydrogen. The functions comprising a labile hydrogen may be alcohol, primary or secondary amine or thiol functions, leading to, after reaction with the diisocyanate functions, polyurethanes, polyureas and polythioureas, respectively. The term “polyurethane” encompasses these three types of polymers: polyurethanes per se, polyureas, polythioureas, and copolymers thereof.

A first type of compound involved in the preparation of the polyurethane of formula (I) is a compound comprising at least one unit comprising an amine function. This compound may be multifunctional, for example, difunctional, i.e., the compound comprises two labile hydrogen atoms borne, for example, by a hydroxyl, primary amine, secondary amine, or thiol function. Mixtures of multifunctional and difunctional compounds in which the percentage of multifunctional compounds is low may also be used.

As mentioned above, this compound may comprise more than one unit comprising an amine function, for example, a polymer bearing a repetition of the unit comprising an amine function.

Compounds of this type may be represented by one of the following formulae:
HZ—(P)ₙ—ZH
or
HZ—(P′)ₚ—ZH
wherein Z, P, P′, n and p are as defined above.

Examples of compounds comprising an amine function include N-methyidiethanolamine, N-tert-butyidiethanolamine, and N-sulfoethyldiethanolamine.

The second compound involved in the preparation of the polyurethane of formula (I) is a diisocyanate of the formula:
O═C═N—R₄—N═C═O
wherein R₄ is as defined above.

Examples include methylenediphenyl diisocyanate, methylenecyclohexane diisocyanate, isophorone diisocyanate, toluene diisocyanate, naphthalene diisocyanate, butane diisocyanate, and hexane diisocyanate.

A third compound involved in the preparation of the polyurethane of formula (I) is a hydrophobic compound intended to form the terminal hydrophobic groups of the polymer of formula (I).

This compound comprises a hydrophobic group and a function comprising a labile hydrogen, for example a hydroxyl, primary or secondary amine, or thiol function.

For example, this compound may be a fatty alcohol such as stearyl alcohol, dodecyl alcohol, or decyl alcohol. When this compound comprises a polymeric chain, it may be, for example, alpha-hydroxylated hydrogenated polybutadiene.

The hydrophobic group of the polyurethane of formula (I) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as, but not limited to, a halide or a sulfate.

The cationic associative polyurethane may also comprise a hydrophilic block, provided by a fourth type of compound involved in the preparation of the polymer. The compound may be multifunctional, e.g., difunctional. It is also possible to have a mixture in which the percentage of multifunctional compound is low.

The functions comprising a labile hydrogen may be chosen from alcohol, primary or secondary amine, and thiol functions, resulting in a compound that is a polymer terminated at the chain ends with one of these functions comprising a labile hydrogen.

By way of example, when Y is not a polymer, it may be chosen from ethylene glycol, diethylene glycol and propylene glycol.

When Y is a hydrophilic polymer, it may be derived from, for example, polyethers, sulfonated polyesters and sulfonated polyamides, or a mixture of these polymers. The hydrophilic compound may be a polyether such as a poly(ethylene oxide) or poly(propylene oxide).

The hydrophilic group Y in formula (I) is optional. The units comprising a quaternary amine or protonated function may suffice to provide the solubility or water-dispersibility required to use this type of polymer in an aqueous solution. However, in certain embodiments, the cationic associative polyurethanes do comprise a hydrophilic group Y.

Anionic Associative Polyurethanes

An example of an anionic associative polyurethane that may be used is an acrylic terpolymer that is soluble or swellable in alkalis, comprising:
(a) from 20% to 70% by weight, for example from 25% to 55% by weight, of a carboxylic acid comprising α, β-monoethylenic unsaturation;
(b) from 20% to 80% by weight, for example from 30% to 65% by weight, of a non-surfactant monomer comprising monoethylenic unsaturation, which is different from the carboxylic acid in (a); and
(c) from 0.5% to 60% by weight, for example 10% to 50% by weight, of a nonionic urethane monomer which is the product of reaction of a monohydric nonionic surfactant with a monoisocyanate comprising monoethylenic unsaturation.

The carboxylic acid comprising α,β-monoethylenic unsaturation in (a) can be chosen from many acids, for example, from acrylic acid, methacrylic acid, itaconic acid and maleic acid, for example methacrylic acid. A large proportion of acid is useful to give a polymer structure which dissolves and gives a thickening effect by reaction with an alkaline compound such as sodium hydroxide, alkanolamines, aminomethylpropanol, and aminomethylpropanediol.

The terpolymer may also comprise a large proportion, indicated above, of a monomer (b) comprising monoethylenic unsaturation which has no surfactant properties. Monomers which may be used include those which give polymers that are water-insoluble when they are homopolymerized, for example, C₁-C₄ alkyl acrylates and methacrylates such as methyl acrylate, ethyl acrylate and butyl acrylate, or corresponding methacrylates, for example, methyl and ethyl (meth)acrylates. Other monomers which may be used are styrene, vinyltoluene, vinyl acetate, acrylonitrile and vinylidene chloride. Non-reactive monomers may be used, such monomers being those in which the single ethylenic group is the only group which is reactive under the polymerization conditions. However, monomers which comprise groups that are reactive under the action of heat may also be used, such as hydroxyethyl acrylate.

The monohydric nonionic surfactants used to obtain the nonionic urethane monomer (c) are well known and are generally alkoxylated hydrophobic compounds comprising an alkylene oxide forming the hydrophilic part of the molecule. The hydrophobic compounds generally comprise an aliphatic alcohol or an alkylphenol in which a carbon chain comprising at least six carbon atoms constitutes the hydrophobic part of the surfactant.

Monohydric nonionic surfactants that may be used include those having the formula: 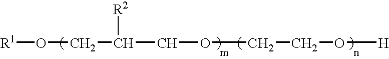
wherein R¹ is a C₆-C₃₀ alkyl or C₈-C₃₀ aralkyl group, R² is a C₁-C₄ alkyl group, n is an average number ranging from 5 to 150 and m is an average number ranging from 0 to 50, with the condition that n is at least as large as m and that the sum of n and m ranges from 5 to 150.

C₆-C₃₀ alkyl groups that may be used include dodecyl and C₁₈-C₂₆ alkyl radicals. Aralkyl groups that may be used include (C₈-C₁₃)alkylphenyl groups. In some embodiments, R² is a methyl group.

The monoisocyanate comprising monoethylenic unsaturation which is used to form the nonionic urethane monomer (c) may be chosen from a wide variety of compounds. A compound comprising any copolymerizable unsaturation such as acrylic or methacrylic unsaturation may be used. An allylic unsaturation imparted by allyl alcohol may also be used. Examples of monoethylenic monoisocyanates include α,α-dimethyl-m-isopropenyl-benzylisocyanate and methylstyrene-isopropylisocyanate.

The acrylic terpolymer defined above may be obtained by aqueous emulsion copolymerization of the components (a), (b) and (c) which is known and described in European Patent Application 173,109 A.

Examples of anionic associative polyurethanes that may be used include copolymers of methacrylic or acrylic acid comprising at least one C₁₋₃₀ alkyl (meth)acrylate unit and a urethane unit substituted with a fatty chain, for example, the methacrylic acid/methyl methacrylate/methylstyrene-isopropyl isocyanate/behenyl alcohol polyethoxylated copolymer (comprising 40 ethoxy units) sold under the brand name Viscophobe® DB 1000 by the company Union Carbide.

Nonionic Associative Polyurethanes

Nonionic associative polyurethanes that may be used include polyurethane polyethers comprising in their chain both polyoxyethylenated hydrophilic blocks and hydrophobic blocks that may be aliphatic sequences alone and/or cycloaliphatic and/or aromatic sequences.

The polyurethane polyethers may comprise at least two hydrocarbon-based lipophilic chains having from 6 to 30 carbon atoms, separated by a hydrophilic block, the hydrocarbon-based chains optionally being pendent chains, or chains at the end of the hydrophilic block. One or more pendent chains may be included. In addition, the polymer may comprise a hydrocarbon-based chain at one end or at both ends of a hydrophilic block.

The polyurethane polyethers may be multiblock, for example, in triblock form. Hydrophobic blocks may be at each end of the chain, e.g., triblock copolymer with a hydrophilic central block, or distributed both at the ends and in the chain, e.g., multiblock copolymer. These polymers may also be graft polymers or starburst polymers.

The nonionic fatty-chain polyurethane polyethers may be triblock copolymers in which the hydrophilic block is a polyoxyethoxylated chain having from 50 to 1000 ethoxylated groups. The nonionic polyurethane polyethers may comprise a urethane linkage between the hydrophilic blocks.

The nonionic fatty-chain polyurethane polyethers also include those in which the hydrophilic blocks are linked to the lipophilic blocks via other chemical bonds.

Examples of nonionic fatty-chain polyurethane polyethers that may be used include Rheolate 205 comprising a urea function, sold by the company Rheox, Rheolate 208, 204, and 212, and Acrysol® RM 184.

The product Elfacos T210 comprising a C₁₂₋₁₄ alkyl chain and the product Elfacos® T212 comprising a C₁₈ alkyl chain, from Akzo may also be used.

The product DW 1206B from Rohm & Haas comprising a C₂₀ alkyl chain and a urethane linkage, sold at a solids content of 20% in water, may also be used.

It is also possible to use solutions or dispersions of these polymers, such as dispersions in water or in an aqueous-alcoholic medium. Examples of such polymers include Rheolate® 255, Rheolate® 278, and Rheolate® 244 sold by the company Rheox. The products DW 1206F and DW 1206J sold by the company Rohm & Haas may also be used.

Polyurethane polyethers that may be used also include those described in the article by G. Fonnum, J. Bakke and Fk. Hansen-Colloid Polym. Sci 271, 380-389 (1993).

Examples of nonionic associative polyurethanes that may be used include polyurethane polyethers that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold by the company Rohm & Haas under the names Aculyn® 46 and Aculyne 44. Aculyn® 46 is a polycondensate of polyethylene glycol comprising 150 to 180 mol of ethylene oxide, of stearyl alcohol and of methylenebis(4-cyclohexylisocyanate) (SMDI), at 15% by weight in a matrix of maltodextrin (4%) and water (81%); Aculyn® 44 is a polycondensate of polyethylene glycol containing 150 to 180 mol of ethylene oxide, of decyl alcohol and of methylenebis(4-cyclohexyl-isocyanate) (SMDI), at 35% by weight in a mixture of propylene glycol (39%) and water (26%).

The polyurethane associative polymers used in the compositions may be present in an amount ranging from 0.01% to 10% by weight relative to the total weight of the dye composition, for example, from 0.1% to 5% by weight.

Alcohol Oxidase Enzymes

The alcohol oxidase enzymes used in the dye compositions disclosed herein may belong to the E.C. 1.1.3 class using enzyme nomenclature (see Enzyme Nomenclature, Academic Press Inc; 1992).

The enzymes may be chosen from primary alcohol oxidases (EC 1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7), also known as aryl alcohol oxidases.

In some embodiments, the enzyme used in the compositions may be a primary alcohol oxidase (EC 1.1.3.13).

Alcohol oxidase enzymes form a class of 2-electron oxidoreductase enzymes. The alcohol oxidase enzymes used in the presently disclosed dye compositions may be derived from extracts of plants, of animals, of micro-organisms (bacterium, fungus, yeast, microalga or virus), of differentiated or undifferentiated cells, obtained in vivo or in vitro, unmodified or genetically modified, or synthetic (e.g., obtained by chemical or biotechnological synthesis).

The alcohol oxidase enzymes may also be derived from one of the following species: Rhodococcus erythropolis, Pseudomonas pseudoalcaligenes which are bacteria, Aspergillus niger, Kamagataella pastoris, Phanerochaete chrysosporium, Polyporus obtusus, Hansenula polymorpha, Poria contigua, Penicillium simplicissimum, Pleurotus pulmonarius (fungi), Pichia sp. (pastoris, methanolica, angusta) and Candida sp. (boidinii, albicans, tropicalis) (yeasts), Pinus strobus, which is a species of plant origin, Gastropode mollusc and Manduca sexta, which are of animal origin.

In some embodiments, the enzyme used in the compositions is an oxidase alcohol derived from Pichia pastoris.

The concentration of alcohol oxidase enzyme used in the dye compositions may range from 0.05% to 20% by weight relative to the total weight of the composition, for example from 0.1% to 10% or from 0.5% to 8% by weight relative to the weight of this composition.

The enzymatic activity of the alcohol oxidase enzymes may be defined from the oxidation of the donor under aerobic conditions. One unit (U) corresponds to the amount of enzyme leading to the generation of 1 μmol of hydrogen peroxide per minute at a given pH and at a temperature of 25° C.

The amount of alcohol oxidase may range from 10³ U to 10⁵ U, for example, from 2×10³ U to 5×10⁴ U, per 100 g of dye composition.

The substrate or substrates for the enzyme are also known as donors for the enzyme.

The substrates for the enzyme used in the compositions may be an alcohol chosen from primary alcohols, secondary alcohols, long-hydrocarbon-chain alcohols, and aromatic alcohols. For example, donors for primary alcohol oxidases include primary alcohols having from 1 to 6 carbon atoms; donors for aryl alcohol oxidases include benzyl alcohol, 4-tert-butylbenzyl alcohol, 3-hydroxy-4-methoxybenzyl alcohol, veratryl alcohol, 4-methoxybenzyl alcohol and cinnamyl alcohol; 2,4-hexadien-1-ol may also be used as a donor for aryl alcohol oxidases.

The enzyme substrate may also be a compound bearing at least one aliphatic or aromatic alcohol function, suitable for reacting with the enzyme used. The compound bearing at least one aliphatic or aromatic alcohol function may be an oxidation dye precursor or a cosmetically acceptable adjuvant, for example a polymer, a surfactant or a preserving agent bearing at least one alcohol function. In some embodiments, the enzyme substrate may be an oxidation dye precursor bearing at least one aliphatic or aromatic alcohol function. For example, N-(β-hydroxypropyl)-para-phenylenediamine, which bears a primary alcohol function, may serve as the oxidation base and as the substrate for the alcohol oxidase. Similarly, oxidation couplers, such as meta- and para-aminophenol, may fulfill the two functions. Such precursors are described below. In these embodiments, the use of other substrates for the enzyme is optional.

Thus, the present disclosure provides compositions for dyeing keratin fibers, including human keratin fibers such as the hair, comprising, in a medium that is suitable for dyeing, at least the following compounds: at least one oxidation dye precursor; at least one alcohol oxidase enzyme; at least one enzyme substrate, bearing an alcohol function, and at least one polyurethane associative polymer, the substrate optionally being substituted (i.e., replaced) totally or partially with the at least one oxidation dye precursor in the case where it bears at least one aliphatic or aromatic alcohol function.

The use of the compositions disclosed herein makes it possible to reduce the risks associated with the handling of hydrogen peroxide. Furthermore, the concentration of preserving agents in the compositions may be reduced by providing compounds comprising an alcohol function that also have preserving properties.

Generally, the concentration of the substrate or substrates for the enzyme ranges from 0.01% to 60% by weight relative to the total weight of the composition, for example, from 0.05% to 30% by weight relative to the total weight of the composition.

Oxidation Dye Precusors

As used herein, the term “oxidation dye precursor” means both oxidation bases and oxidation couplers. In some embodiments, only oxidation bases are included in the presently disclosed compositions.

The oxidation bases useful in the compositions disclosed herein may be chosen, for example, from para-phenylenediamines, bis(phenyl)alkylenediamines, para-aminophenols, ortho-aminophenols, heterocyclic bases, and addition salts thereof.

Para-phenylenediamines include, but are not limited to, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenytenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-2-methylaniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylened amine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino- . 5-aminotoluene, 3-hydroxy-1-(4′-aminophenyl)pyrrolidine, and acid addition salts thereof.

In some embodiments, the para-phenylenediamines used in the compositions are chosen from para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N, N-bis(β-hydroxyethyl)-para-phenylenediamine, 2-chloro-para-phenylenediamine, 2-β-acetylaminoethyloxy-para-phenylenediamine, and acid addition salts thereof.

Bis(phenyl)alkylenediamines include, for example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and acid addition salts thereof.

Para-aminophenols include, for example, para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, 4-amino-2,6-dichlorophenol, 4-amino-6[((5′-amino-2′-hydroxy-3′-methyl)phenyl)methyl]-2-methylphenol, bis(5′-amino-2′-hydroxy)phenylmethane, and acid addition salts thereof.

Ortho-aminophenols include, for example, 2-aminophenol, 2-amino-5-methylphenol, 2-amino-6-methylphenol, 5-acetamido-2-aminophenol, and acid addition salts thereof.

Heterocyclic bases include, for example, pyridine derivatives, pyrimidine derivatives, and pyrazole derivatives.

Pyridine derivatives include, for example, the compounds described, for example, in GB Patent Nos. 1,026,978 and 1,153,196, as well as 2,5-diaminopyridine, 2-(4-methoxyphenyl)amino-3-aminopyridine, 3,4-diaminopyridine, and acid addition salts thereof.

Other pyridine oxidation bases that may be used include 3-aminopyrazolo-[1,5-a]pyridine oxidation bases and addition salts thereof described, for example, in French Patent Application No. 2,801,308. Examples include pyrazolo[1,5-a]pyrid-3-ylamine; 2-acetylaminopyrazolo[1,5-a]pyrid-3-ylamine; 2-morpholin-4-ylpyrazolo[1,5-a]pyrid-3-ylamine; 3-aminopyrazolo[1,5-a]pyrid in-2-carboxylic acid; 2-methoxypyrazolo[1,5-a]pyrid-3-ylamine; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyrid ine; 3,4-diaminopyrazolo[1,5-a]pyridine; pyrazolo[1,5-a]pyrid-3,7-diamine; 7-morpholin-4-ylpyrazolo[1,5-a]pyrid-3-ylamine; pyrazolo[1,5-a]pyrid-3,5-diamine; 5-morpholin-4-ylpyrazolo[1,5-a]pyrid-3-ylamine; 2-[(3-aminopyrazolo[1,5-a]pyrid-5-yl)-(2-hydroxyethyl)amino]ethanol; 2-[(3-aminopyrazolo[1,5-a]pyrid-7-yl)-(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyridine-6-ol; 3-aminopyrazolo[1,5-a]pyrid-7-ol; and acid addition salts thereof.

Pyrimidine derivatives include the compounds described, for example, in patents DE 2,359,399; JP 88-169,571; JP 05,163,124; EP 0,770,375; and patent application WO 96/15765, such as 2,4,5,6rtetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, addition salts thereof, and tautomeric forms thereof, when a tautomeric equilibrium exists.

Pyrazole derivatives include the compounds described in patents DE 3,843,892 and DE 4,133,957 and patent applications WO 94/08969, WO 94/08970, FR-A-2,733,749 and DE 19,543,988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1 -tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl4-methylaminopyrazole, 3,5-diamino4-(β-hydroxyethyl)amino-1-methylpyrazole, and addition salts thereof.

Generally, the concentration of the oxidation bases ranges from 0.0001% to 20%, for example, from 0.005% to 6% by weight, relative to the total weight of the composition.

Oxidation couplers include, for example, meta-phenylenediamines, meta-aminophenols, meta-diphenols, naphthalenic couplers, heterocyclic couplers, and addition salts thereof.

Examples of oxidation couplers include 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethyl-amino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, and addition salts thereof.

Generally, the concentration of the oxidation coupler or couplers ranges from 0.0001% to 20%, for example from 0.005% to 6%, by weight relative to the total weight of the composition.

In general, the acid addition salts that may be used for the oxidation bases and couplers may be chosen from the hydrochlorides, hydrobromides, sulphates, citrates, succinates, tartrates, lactates, tosylates, benzenesulphonates, phosphates, and acetates.

The base addition salts that may be used may be chosen from, for example, addition salts with sodium hydroxide, potassium hydroxide, ammonia, amines, and alkanolamines.

Direct Dyes

The dye compositions may also comprise at least one direct dye, which may be chosen from neutral, acidic, and cationic nitrobenzene dyes; neutral, acidic or catiQnic azo direct dyes; neutral, acidic, and cationic quinone such as anthraquinone direct dyes, azine direct dyes, methine, azomethine, triarylmethane, and indoamine direct dyes, and natural direct dyes. These additional direct dyes may be chosen from cationic direct dyes and natural direct dyes.

Cationic direct dyes that may be used include cationic azo direct dyes described in patent applications WO 95/15144, WO 95/01772 and EP 714,954.

These compounds include 1,3-dimethyl-2-[[4-(dimethylamino)phenyl]azo]-1H-imidazolium chloride, 1,3-dimethyl-2-[(4-aminophenyl)azo]-1H-imidazolium chloride, and 1-methyl4-[(methylphenylhydrazono)methyl]pyridinium methyl sulphate.

Natural direct dyes that may be used include lawsone, juglone, alizarin; purpurin, carminic acid, kermesic acid, purpurogallin, protocatechaldehyde, indigo, isatin, curcumin, spinulosin, and apigenidin. It is also possible to use extracts or decoctions comprising these natural dyes, for example, henna-based poultices and extracts.

The at least one direct dye, if present, is present in the composition in an amount ranging from 0.001% to 20% by weight, for example from 0.005% to 10% by weight, relative to the total weight of the composition.

Adjuvants

The dye compositions disclosed herein may further comprise various additional adjuvants other than polyurethane associative polymers. These additional agents are conventionally used in compositions for dyeing the hair, including, for example, antioxidants, penetrating agents, sequestering agents, fragrances, buffers, dispersants, surfactants, conditioners such as, for example, volatile or non-volatile, modified or unmodified silicones, cationic polymers, cations, film-forming agents, ceramides, preserving agents, opacifiers, vitamins, and provitamins.

These adjuvants are generally each present in an amount ranging from 0.01% to 20% by weight relative to the total weight of the composition.

Needless to say, a person skilled in the art will take care to select optional additional compounds such that the advantageous properties intrinsically associated with the oxidation dye compositions are not, or are not substantially, adversely affected by the envisaged additions.

The medium that is suitable for dyeing, also known as the dye support, generally comprises water or a mixture of water and at least one organic solvent to dissolve the compounds that would not be sufficiently water-soluble. As appropriate, this solvent may be an enzyme substrate such as ethanol or isopropanol. It may also be not be an enzyme substrate such as polyol ethers, for example, 2-butoxyethanol, propylene glycol, propylene glycol monomethyl ether, diethylene glycol monomethyl ether, monoethyl ether, phenoxyethanol, and mixtures thereof.

The solvents may be present in proportions ranging from 1% to 40% by weight relative to the total weight of the dye composition, for example ranging from 5% to 30% by weight.

The pH of the presently disclosed dye compositions generally ranges from 6 to 11, for example, from 7 to 10. It may be adjusted to a desired value using acidifying or basifying agents usually used in the dyeing of keratin fibers, or alternatively using standard buffer systems.

Acidifying agents that may be used include, for example, mineral and organic acids, for example hydrochloric acid, orthophosphoric acid, sulphuric acid, carboxylic acids, e.g., acetic acid, tartaric acid, citric acid and lactic acid, and sulphonic acids.

Basifying agents that may be used include, for example, aqueous ammonia, alkyl metal carbonates, alkanolamines such as monoethanolamine, diethanolamine and triethanolamine, and derivatives thereof, sodium hydroxide, potassium hydroxide, and the compounds of formula (III): 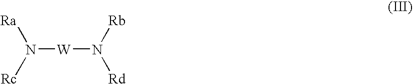
wherein W is a propylene residue optionally substituted with a hydroxyl group or a C₁-C₄ alkyl radical; Rₐ, R_{b}, R_{c} and R_{d}, which may be identical or different, are each chosen from a hydrogen atom, a C₁-C₄ alkyl, and a C₁-C₄ hydroxyalkyl radical.

The dye composition may be in various forms, such as in the form of thickened liquids, creams, gels, and in any other form that is suitable for dyeing keratin fibers, such as human hair.

When the oxidation dyes and the alcohol oxidase or oxidases are present in the same ready-to-use composition, the composition is optionally free of oxygen gas, so as to avoid any premature oxidation of the oxidation dye or dyes.

The present disclosure also provides processes for dyeing keratin fibers, including human keratin fibers such as the hair, such that at least one dye composition described above is applied to the fibers for a duration that is sufficient to develop the desired coloration.

The color is then revealed by bringing together the alcohol oxidase enzyme and its substrate in the presence of oxygen.

The composition is applied to the keratin fibers. After leaving it to act for a sufficient time to develop the desired color, for example, from 3 to 60 minutes or from 5 to 40 minutes, the keratin fibers are rinsed, washed with shampoo, rinsed again, and dried.

When the dye compositions are compositions in ready-to-use form, they comprise, in a medium that is suitable for dyeing keratin fibers, at least one oxidation dye precursor, at least one alcohol oxidase enzyme, at least one enzyme substrate, and at least one polyurethane associative polymer, and the mixture is then stored in anaerobic form, free of oxygen gas.

In some embodiments, the process includes a preliminary step comprising separately storing a composition (A) comprising, in a medium that is suitable for dyeing keratin fibers, at least one oxidation dye precursor, and a composition (B) comprising, in a medium that is suitable for dyeing keratin fibers, at least one alcohol oxidase enzyme, the composition (A) and/or the composition (B) comprising at least one enzyme substrate, and the composition (A) and/or the composition (B) comprising at least one polyurethane associative polymer, and then in mixing together the compositions (A) and (B) at the time of use before applying the mixture to the keratin fibers.

In some embodiments, the process includes a preliminary step that comprises separately storing a composition (A) comprising, in a medium that is suitable for dyeing keratin fibers, at least one oxidation dye precursor, at least one enzyme substrate, and at least one polyurethane associative polymer and a composition (B) comprising, in a medium that is suitable for dyeing keratin fibers, at least one alcohol oxidase enzyme, and then in mixing together the compositions (A) and (B) at the time of use before applying the mixture to the keratin fibers.

The color may be revealed at acidic, neutral or alkaline pH. In the case where the process is performed using a composition (A) comprising at least one oxidation dye precursor, at least one enzyme substrate and at least one polyurethane associative polymer and a composition (B) comprising at least one alcohol oxidase enzyme, the enzyme may be added to the composition just at the time of use, or it may be used starting with a composition comprising it, applied simultaneously or sequentially to the composition.

Composition B (the oxidizing composition) may further comprise various adjuvants conventionally used in compositions for dyeing the hair and as described above.

The pH of composition B is such that, after mixing with dye composition A, the pH of the resultant composition applied to the keratin fibers may range from 6 to 11, such as from 7 to 10. It may be adjusted to the desired value by means of acidifying or basifying agents usually used in the dyeing of keratin fibers and as described above.

The application of the compositions may be carried out at a temperature ranging from ambient temperature (e.g., 22 or 25° C.) to 220° C., for example, from ambient temperature to 60° C.

The present disclosure also provides multi-compartment devices or dyeing “kits,” in which a first compartment contains the composition (A) as defined above and a second compartment contains the composition (B) as defined above. The devices may be equipped with a means for applying the desired mixture to the hair, such as the devices described in French Patent No. 2,586,913.

The invention is illustrated in greater detail by the example described below. Other than in the example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLE

The following composition was prepared:

| Constituents | Composition 1 |
|---|---|
| Viscophobe ® DB 1000 (Union Carbide) | 1% |
| Ethanol | 25 g |
| para-Phenylenediamine | 3 × 10⁻³ mol |
| meta-Aminophenol | 3 × 10⁻³ mol |
| Alcohol oxidase | 20,000 units |
| 2-Amino-2-methyl-1-propanol | qs pH 7 |
| Distilled water | qs 100 g |

The alcohol oxidase used is sold by the company Biozyme Laboratories in liquid form at a concentration of 1980 units/ml.

The unit U corresponds to the amount of enzyme leading to the generation of 1 μmol of hydrogen peroxide per minute at pH 7.5 (100 mM phosphate buffer) and at a temperature of 25° C.

The above composition was applied to locks of natural grey permanent-waved hair having 90% white hair, and left to act for 30 minutes. The bath ratio was set at 5. The alcohol oxidase was added extemporaneously. The hair was then rinsed, washed with a standard shampoo and then dried.

The hair was dyed in shades of khaki green.

1. A composition for dyeing keratin fibers comprising, in a medium suitable for dyeing,
at least one oxidation dye precursor,
at least one alcohol oxidase enzyme,
at least one enzyme substrate bearing an alcohol function for said at least one enzyme, and
at least one polyurethane associative polymer,
wherein the at least one enzyme substrate may be totally or partially substituted by the oxidation dye precursor in the case where the at least one precursor bears at least one aromatic or aliphatic alcohol functional group.

2. The composition according to claim 1, wherein the keratin fibers are human hair.

3. The composition according to claim 1, wherein the polyurethane associative polymer is a cationic polyurethane of formula (I):
R—X—(P)ₙ—[L—(Y)ₘ]ᵣ—L′—(P′)ₚ—X′—R′
wherein:
R and R′, which may be identical or different, are each chosen from a hydrophobic group and a hydrogen atom;
X and X′, which may be identical or different, are each chosen from a group comprising an amine function optionally bearing a hydrophobic group, or alternatively from a group L″;
L, L′ and L″, which may be identical or different, are each chosen from a group derived from a diisocyanate;
P and P′, which may be identical or different, are each chosen from a group comprising an amine function optionally bearing a hydrophobic group;
Y is a hydrophilic group;
r is an integer ranging from 1 to 100;
n, m and p, independently of each other, are each integers ranging from 0 to 1000; and
wherein the polymer comprises at least one quaternary or quaternized amine function and at least one hydrophobic group.

4. The composition according to claim 3, wherein R and R are the only hydrophobic groups of the polymer.

5. The composition according to claim 3, wherein R and R′ each are independently chosen from a hydrophobic group, X and X′ each are a L″ group; and n and p are each integers ranging from 1 to 1000.

6. The composition according to claim 3, wherein R and R′ each are independently chosen from a hydrophobic group, X and X′ are each L″, n is 0, and p is 0.

7. The composition according to claim 3 wherein R and R′ each are independently chosen from a hydrophobic group, X and X′ each are chosen from a group comprising a quaternary amine, n is 0, and p is 0.

8. The composition according to claim 3, wherein the number-average molecular mass of the polymer ranges from 400 to 500,000.

9. The composition according to claim 8, wherein the number-average molecular mass of the polymer ranges from 1000 to 300,000.

10. The composition according to claim 3, wherein R and R′ are each chosen from a radical or polymer comprising a saturated or unsaturated, linear or branched hydrocarbon-based chain, optionally comprising at least one heteroatom, at least one radical comprising a perfluoro chain, or at least oneradical comprising a silicone chain.

11. The composition according to claim 3, wherein X is chosen from: 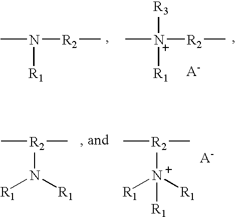
and X′ is chosen from: 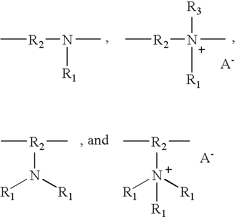
wherein:
R₂ is chosen from a linear or branched alkylene radical having from 1 to 20 carbon atoms, optionally comprising a saturated or unsaturated ring, and from an arylene radical, wherein at least one of the carbon atomsis optionally replaced with a heteroatom chosen from N, S, O, and P;
R₁ and R₃, which may be identical or different, are each chosen from linear and branched C₁-C₃₀ alkyl radicals, linear and branched C₁-C₃₀ alkenyl radical and aryl radicals, optionally comprising at least one heteroatom chosen from N, S, O and P; and
A⁻ is a physiologically acceptable counterion.

12. The composition according to claim 3 wherein the groups L, L′ and L″ each are independently chosen from the formula: 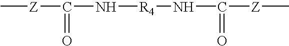
wherein:
Z is chosen from —O—, —S—, and —NH—; and
R₄ is chosen from a linear or branched alkylene radical having from 1 to 20 carbon atoms and optionally comprising a saturated or unsaturated ring, and from an arylene radical optionally comprising at least one heteroatom.

13. The composition according to claim 12, wherein R₄ comprises 1, 2 or 3 heteroatoms.

14. The composition according to claim 3, wherein the groups P and P′, which may be identical or different, are each independently chosen from the following formulae: 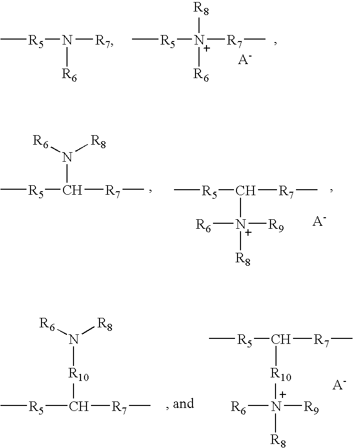
wherein:
R₅ and R₇, which may be identical or different, are each chosen from linear and branched alkylene radicals having from 1 to 20 carbon atoms, optionally comprising a saturated or unsaturated ring, and arylene radicals, wherein at least one of the carbon atoms is optionally replaced with a heteroatom chosen from N, S, O, and P;
R₆, R₈ and R₉, which may be identical or different, are each chosen from linear and branched C₁-C₃₀ alkyl radicals, linear and branched C₁-C₃₀ alkenyl radicals and aryl radicals, optionally comprising at least one heteroatom chosen from N, S, O and P;
R₁₀ is a linear or branched, optionally unsaturated alkylene group optionally comprising at least one heteroatom chosen from N, O, S and P; and
A⁻ is a physiologically acceptable counterion.

15. The composition according to claim 3, wherein Y is chosen from a group derived from ethylene glycol, from diethylene glycol, from propylene glycol, or a group derived from a polymer chosen from polyethers, sulphonated polyesters, sulphonated polyamides, and mixtures thereof.

16. The composition according to claim 1, wherein the associative polymer is an acrylic terpolymer anionic polyurethane comprising:
a) from 20% to 70% by weight of an α,β-monoethylenically unsaturated carboxylic acid;
b) from 20% to 80% by weight of a non-surfactant monoethylenically unsaturated monomer other than a) and c) from 0.5% to 60% by weight of a nonionic urethane monomer, which is the product of reaction of a monohydric nonionic surfactant with a monoethylenically unsaturated monoisocyanate.

17. The composition according to claim 16, wherein the associative polymer is a methacrylic acid/methyl methacrylate/methylstyrene-isopropyl isocyanate/polyethoxylated behenyl alcohol copolymer comprising 40 ethoxy units.

18. The composition according to claim 1, wherein the polyurethane associative polymer is a nonionic polyether-polyurethane, obtained by polycondensation of (i) at least one polyethylene glycol comprising from 150 to 180 mol of ethylene oxide, (ii) stearyl alcohol or decyl alcohol, and (iii) at least one diisocyanate.

19. The composition according to claim 18, wherein the nonionic polyether-polyurethane is a polyethylene glycol polycondensate comprising 150 to 180 mol of ethylene oxide, of stearyl alcohol and of methylenebis(4-cyclohexyl isocyanate) or a polyethylene glycol polycondensate comprising 150 to 180 mol of ethylene oxide, of decyl alcohol and of methylenebis(4-cyclohexyl isocyanate).

20. The composition according to claim 1, wherein the polyurethane associative polymer is present in an amount ranging from 0.01% to 10% by weight relative to the total weight of the dye composition.

21. The composition according to claim 20, wherein the polyurethane associative polymer is present in an amount ranging from 0.1% to 5% by weight relative to the total weight of the dye composition.

22. The composition according to claim 1, wherein the alcohol oxidase enzyme is chosen from primary alcohol oxidases (EC 1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38), and aromatic alcohol oxidases (EC 1.1.3.7).

23. The composition according to claim 21, wherein the alcohol oxidase enzyme is derived from Rhodococcus erythropolis, Pseudomonas pseudoalcaligenes, Aspergillus niger, Kamagataella pastoris, Phanerochaete chrysosporium, Polyporus obtusus, Hansenula polymorpha, Poria contigua, Penicillium simplicissimum, Pleurotus pulmonarius, Pichia pastoris, Pichia methanolica, Pichia angusta, Candida boidinii, Candida albicans, Candida tropicalis, Pinus strobus, Gastropode mollusc, or Manduca sexta.

24. The composition according to claim 22 wherein the concentration of alcohol oxidase enzyme ranges from 0.05% to 20% by weight relative to the total weight of the composition.

25. The composition according to claim 24, wherein the concentration of alcohol oxidase enzyme ranges from 0.5% to 8% by weight relative to the total weight of the composition.

26. The composition according to claim 1, wherein the amount of alcohol oxidase ranges from 10³ U to 10⁵ U per 100 g of dye composition.

27. The composition according to claim 26, wherein the amount of alcohol oxidase ranges from 2×10³ U to 5×10⁴ U per 100 g of dye composition.

28. The composition according to claim 1, wherein the enzyme substrate is an alcohol chosen from primary alcohols, secondary alcohols, long-hydrocarbon-chain alcohols, and aromatic alcohols.

29. The composition according to claim 1, wherein the concentration of the enzyme substrate ranges from 0.01 % to 60% by weight relative to the total weight of the composition.

30. The composition according to claim 29, wherein the concentration of the enzyme substrate ranges from 0.05% to 30% by weight relative to the total weight of the composition.

31. The composition according to claim 1, wherein the at least one oxidation dye precursor is an oxidation base chosen from para-phenylenediamines, bis(phenyl)alkylenediamines, para-aminophenols, ortho-aminophenols, heterocyclic bases, and addition salts thereof.

32. The composition according to claim 31, wherein the concentration of the oxidation base ranges from 0.0001% to 20% by weight relative to the total weight of the composition.

33. The composition according to claim 1, wherein the oxidation dye precursor is an oxidation coupler chosen from meta-phenylenediamines, meta-aminophenols, meta-diphenols, naphthalenic couplers, heterocyclic couplers, and addition salts thereof.

34. The composition according to claim 33, wherein the concentration of the oxidation coupler ranges from 0.0001 % to 20% by weight relative to the total weight of the composition.

35. The composition according to claim 1, further comprising at least one direct dye chosen from cationic and natural direct dyes.

36. A process for dyeing keratin fibers comprising:
applying to the keratin fibers, for a period sufficient to develop a desired coloration, a composition comprising, in a medium suitable for dyeing,
at least one oxidation dye precursor,
at least one alcohol oxidase enzyme,
at least one enzyme substrate bearing an alcohol function, and
at least one polyurethane associative polymer,
wherein the at least one enzyme substrate may be totally or partially substituted by the oxidation dye precursor in the case where the at least one precursor bears at least one aromatic or aliphatic alcohol functional group.

37. The process according to claim 36, wherein the keratin fibers are human hair.

38. The process according to claim 36, wherein the composition is a ready-to-use composition, and wherein the process further comprises storing the ready-to-use composition in anaerobic form, free of oxygen gas.

39. A process for dyeing keratin fibers comprising:
separately storing a first composition comprising, in a medium that is suitable for dyeing keratin fibers, at least one oxidation dye precursor, and a second composition comprising, in a medium that is suitable for dyeing keratin fibers, at least one alcohol oxidase enzyme, the first composition and/or the second composition comprising at least one enzyme substrate and the first composition and/or the second composition comprising at least one polyurethane associative polymer;
mixing together, at the time of use and before applying this mixture to the keratin fibers, the first and second compositions to form a third composition; and
applying the third composition to the keratin fibers for a period sufficient to develop a desired coloration.

40. The process according to claim 39, wherein the first composition comprises the at least one oxidation dye precursor, the at least one enzyme substrate, and the at least one polyurethane associative polymer and the second composition comprises the at least one alcohol oxidase enzyme.

41. A multi-compartment device comprising
a first compartment comprising a first composition comprising, in a medium that is suitable for dyeing keratin fibers, at least one oxidation dye precursor and
a second compartment comprising a second composition comprising, in a medium that is suitable for dyeing keratin fibers, at least one alcohol oxidase enzyme, wherein the first composition and/or the second composition comprise at least one enzyme substrate and the first composition and/or the second composition comprise at least one polyurethane associative polymer.
